(19) United States
(12) Patent Application Publication
(10) Pub. No.: US 2026/0263060 A1
(43) Pub. Date: Sep. 10, 2026

(54) WOUND TREATMENT SYSTEMS, METHODS, AND DEVICES

(71) Applicant: Defense Medical Technologies, Inc., Tampa, FL (US)

(72) Inventors: Frederick J. Thabet, Boca Raton, FL (US); Ravi Kanagala, Boca Raton, FL (US); Keri George, Calgary (CA)

(73) Assignee: Defense Medical Technologies, Inc., Tampa, FL (US)

(21) Appl. No.: 19/355,721

(22) Filed: Oct. 10, 2025

Related U.S. Application Data
(60) Provisional application No. 63/705,877, filed on Oct. 10, 2024.
(60) Provisional application No. 63/735,179, filed on Dec. 17, 2024.

(51) Int. Cl. A61B 17/00

(52) U.S. Cl. CPC A61B 17/0057; A61B 2017/00557; A61B 2017/00893; A61B 2017/00951

(57) ABSTRACT

Systems, methods, and devices disclosed herein include a wound treatment device for providing immediate treatment to a deep tissue wound. The device includes a compressed gas housing with an elongated body and one or more actuator(s) at a first end. The one or more actuator(s) include an air pump plunger and/or a compressed carbon dioxide actuator. The wound treatment device also includes a balloon insertion assembly operable to couple to a second end of the compressed gas housing. The balloon insertion assembly includes a tamponade balloon which at least partly inflates inside the wound responsive to actuation of the compressed gas housing. Additionally, the wound treatment device comprises a device positioning assembly including a locking balloon mount, and adhesive pad, and/or one or more straps operable to secure the balloon insertion assembly at a target wound location.

### CROSS-REFERENCE TO RELATED APPLICATIONS

This application claims priority to U.S. Provisional Patent Application Ser. No. 63/735,179, filed Dec. 17, 2024 and titled “WOUND TREATMENT SYSTEMS, METHODS, AND DEVICES,” and U.S. Provisional Patent Application Ser. No. 63/705,877, filed Oct. 10, 2024 and titled “WOUND TREATMENT SYSTEMS, METHODS, AND DEVICES,” the entireties of which are incorporated by reference herein.

### BACKGROUND

Wound treatment techniques performed in remote locations away from a clinic setting typically only provide topical treatment or treat only a portion of a wound. These types of treatments do not treat an interior portion of the wound and can be ineffective for treating deeper wounds. When the injury occurs in the field, more substantive treatment, such as internal stitches, are often delayed until the injured person is relocated to the clinic setting. Many injured people lose dangerous amounts of blood immediately following their injury while awaiting transport to the clinic setting.

It is with these observations in mind, among others, that various aspects of the present disclosure were conceived and developed.

### SUMMARY

Systems, methods, and devices disclosed herein can address the aforementioned issues. For instance, a method of treating a wound can include inserting a tamponade balloon of a wound treatment device into a target wound receiving treatment from the wound treatment device; securing the wound treatment device in place at the target wound using a device positioning assembly; and/or inflating the tamponade balloon using an actuator disposed on the wound treatment device.

In some examples, the target wound can occur in a non-human animal, such as a cow or bull. Also the target wound is a result of a fly worm infection, and/or the tamponade balloon can be coated in one or more carrier agents to treat the fly worm infection. The one or more carrier agents can include at least one of a pesticide, a larvicide, a macrocyclic lactone, or an anthelmintic. Also, the non-human animal can include one of cattle, a sheep, a goat, a pig, a dog, a cat, a chicken, a horse, a donkey, a duck, a buffalo, a llama, an alpaca, or a camel. Additionally, the inflating of the tamponade balloon causes one or more treatment agents to be provided into the target wound. The treatment agent can at least one of reduces or prevents a fly worm infection. Furthermore, the wound treatment device includes a balloon cannula housing with an elongated cross-sectional profile corresponding to a shape of the target wound. The wound treatment device can also include a female luer locking port, and/or the method can further include administering one or more insecticide or larvicide agents to the tamponade balloon through the female luer locking port.

In some scenarios, a method of treating a wound includes inserting a tamponade balloon of a wound treatment device into a target wound of a human patient or a non-human patient receiving treatment from the wound treatment device. The tamponade balloon can be part of a balloon insertion assembly. The method can also include securing the wound treatment device in place at the target wound using a device positioning assembly; inflating the tamponade balloon using an actuator disposed on the wound treatment device; and/or treating the target wound with one or more carrier agents disposed on the tamponade balloon.

In some instances, the target wound is of the non-human patient, and/or the one or more carrier agents includes an insecticide and/or a larvicide. Furthermore, the target wound can be a knife wound of the human patient. The balloon insertion assembly can also include an elongated cross-sectional profile which corresponds to a cross-sectional profile of the knife wound.

In some examples, a method of treating a wound includes inserting a tamponade balloon of a wound treatment device into a target wound of a patient receiving treatment from the wound treatment device, the tamponade balloon extending from a balloon cannula housing; securing the wound treatment device in place at the target wound using a device positioning assembly; and/or inflating the tamponade balloon using an actuator disposed on the wound treatment device. The balloon cannula housing can include an elongated cross-sectional profile. Additionally, the method can further include selecting the balloon cannula housing including the elongated cross-sectional profile from a plurality of balloon cannula housings having different cross-sectional profiles. The selecting of the balloon cannula can be based on a shape of the target wound. Moreover, the target wound can include an insect or parasite infection, and/or the inflating of the tamponade balloon can treat the insect or parasite infection.

### BRIEF DESCRIPTION OF THE DRAWINGS

FIGS. 1A and 1B illustrate an example system including a portable wound treatment device.

FIGS. 1C and 1D illustrate an example system including a portable wound treatment device with a compressed gas actuator.

FIGS. 2A and 2B illustrate an example system including a balloon insertion assembly of a portable wound treatment device.

FIGS. 3A-3C illustrate an example system including a device positioning assembly of a portable wound treatment device.

FIGS. 4A and 4B illustrate an example system including an air pump assembly of a portable wound treatment device.

FIGS. 5A-5C illustrate example methods to treat a wound which can be performed with any of the system(s) depicted in FIGS. 1A-4B.

### DETAILED DESCRIPTION

It will be appreciated that numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

The systems, methods, and devices disclosed herein include a wound treatment device for immediate treatment of a wound (e.g., a bullet wound, a knife wound, and so forth) to stop the bleeding. The wound treatment device can include a tamponade balloon which is pushed into the wound, a device positioning assembly to secure the device in place, and/or a compressed gas housing to cause the tamponade balloon to inflate.

The wound treatment device can have a portable, stored configuration, and can fit in a 4″×4″×8″ pouch (e.g., an Individual/Improvised First Aid Kit) and/or medic bag for deployment in the field. Furthermore, the wound treatment device can include one or more redundancies and/or failsafe mechanisms, such as a manual pump system in addition to a compressed carbon dioxide actuation system. Also, the wound treatment device can include a pilot balloon to indicate whether pressure is successfully being applied at the wound site. Accordingly, the disclosed technology can be used in the field to provide immediate treatment to a wound to slow the bleeding until the patient can be transported to clinic or emergency room.

Additionally, the balloon inflation of the Bangalore can be designed with a redundant inflation methodology including an attached inflation syringe as well as a CO₂ powered auxiliary handpiece can be used with various FDA approved hemostatic agents such as Thrombin and/or TRAUMAGEL® (e.g., a plant-based, algae-derived, hemostatic gel).

In some examples, the wound treatment device is designed such that the person who is wounded (e.g., has been shot) can self-administer this life saving technology directly and under their own power. For instance, the wound treatment device can be stored in a fully-assembled and ready-to-use configuration. The application procedure can include a one-stage process and/or a two-stage process for inserting the wound treatment device and inflating the balloon, which can be performed by the patient and/or using one hand. Also, the wound treatment device can include one or more mounting mechanisms, such as a shoulder attachment mount, a belt attachment mount, a pants attachment mount, a vest pock clip, and/or other type of clothing attachment mount such that the device can be mounted to uniform of a patient (e.g., a soldier) prior to use, and can be easily accessed and extracted by the patient for use during the application procedure.

Also, the wound treatment device can include one or more components to integrate its use with a tourniquet, such as a Combat Applications Tourniquet (CAT). In some situations, a method of applying the wound treatment device can include accessing the wound treatment device; exposing the balloon; inserting the balloon into the wound; accessing and/or unfurling the CAT; cutting a slit into the CAT; placing the CAT over the wound treatment device such that the hand pump, luer lock, and/or CO₂ actuator is accessible through the slit; securing the CAT around the injured limb/body part to apply pressure with the CAT; and actuating the hand pump and/or CO₂ actuator to cause the balloon to inflate. Moreover, these different steps/stages of application can be performed by a single person, such as the patient during self-administration, or by a field medic, a second soldier, or another person in proximity to the patient.

Alternatively, the different stages of applying the wound treatment device can be performed by a combination of multiple people, such as the patient and second person (e.g., the field medic, another soldier, a surgeon, and so forth). For instance, the initial stage(s) of accessing the wound treatment device, exposing the balloon, and/or inserting the balloon into the balloon can be performed by the patient as self-administration steps. The patient can apply hand pressure to the wound treatment device following these steps while waiting for a second person to arrive and perform the later stage operations. Then, once the second person arrives, the later stage operation(s) can be performed by the second person, including accessing and/or unfurling the CAT (e.g., mounted to the patient or mounted to the second person); cutting a slit into the CAT; placing the CAT over the wound treatment device such that the hand pump, luer lock, and/or CO₂ actuator is accessible through the slit; securing the CAT around the injured limb/body part to apply pressure with the CAT; and actuating the hand pump and/or CO₂ actuator to cause the balloon to inflate.

Furthermore, it is to be understood that the disclosed systems, methods, and devices can be used with non-human patients, such as animals. For instance, domesticated animals may suffer injuries resulting in an open wound. These injuries can occur from disease, such as a fly worm pest (e.g., new world screwworm, buffalo fly, etc.) which burrows into the flesh of the animal to lay eggs. A caretaker of the animal must extract the fly worm pest, which can leave a wound in the animal. Additional wounds can occur from the feeding activities of the pest itself. As such, the disclosed device can be used to treat these wounds as part of a pest removal procedure, and/or post-removal treatment. Additionally, the balloon of the device can be coated with various carrier agents for specifically treating these types of wounds. The balloon can be coated in an insecticide (e.g., pyrethroids), a larvicide, an antibiotic (e.g., penicillin), macrocyclic lactones (e.g., ivermectin), anthelmintics (e.g., nitroxinil), and/or combinations thereof. This treatment can also be used for animal wounds with other causes (e.g., bites from other animals, collisions with gates/wires, falls, etc.) to prevent an insect infestation in the wound. As discussed herein, the device can be used to treat wounds in cattle (e.g., cows and/or bulls), sheep, goats, pigs, dogs, cats, chickens, horses, donkeys, ducks, buffalo, llamas, alpacas, camels, and so forth. Human patients can also receive treatment for insect and/or parasite infections by using these techniques.

Additional advantages of the technology will become apparent from the detailed description below.

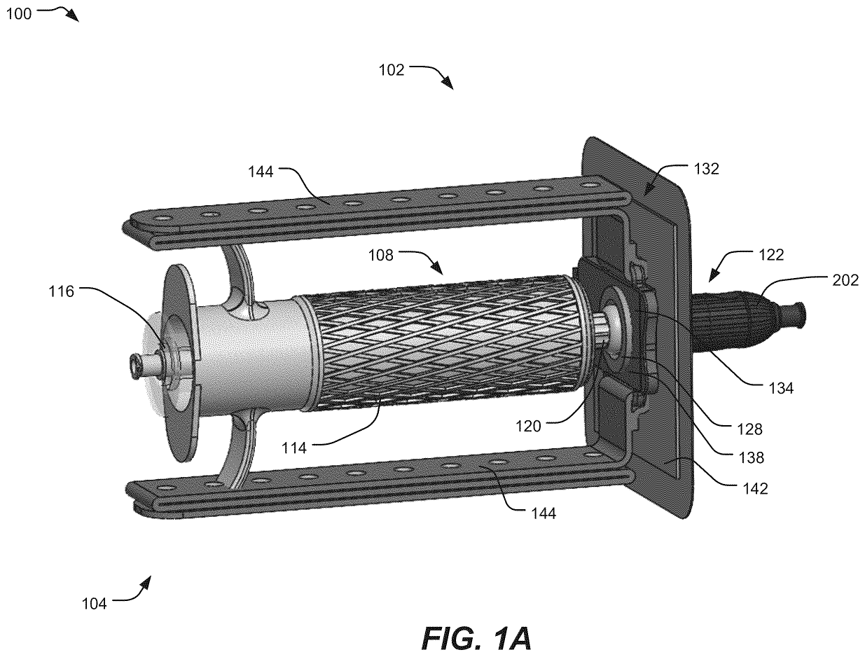

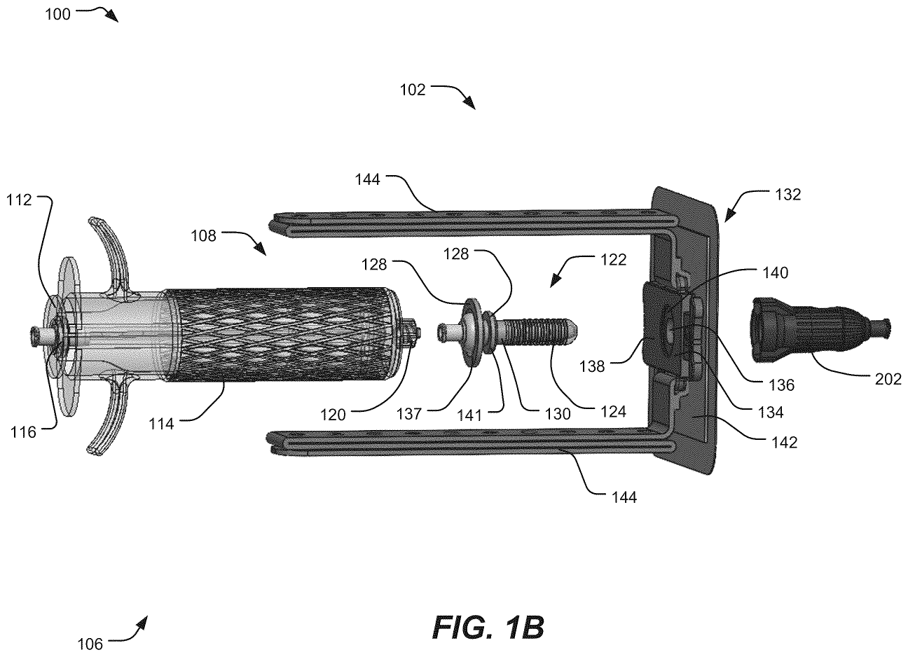

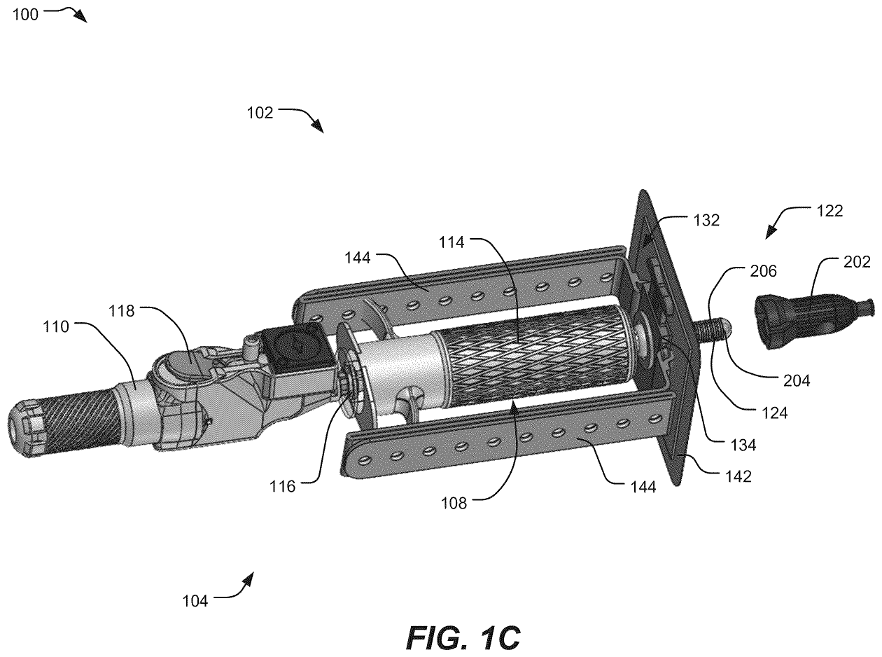

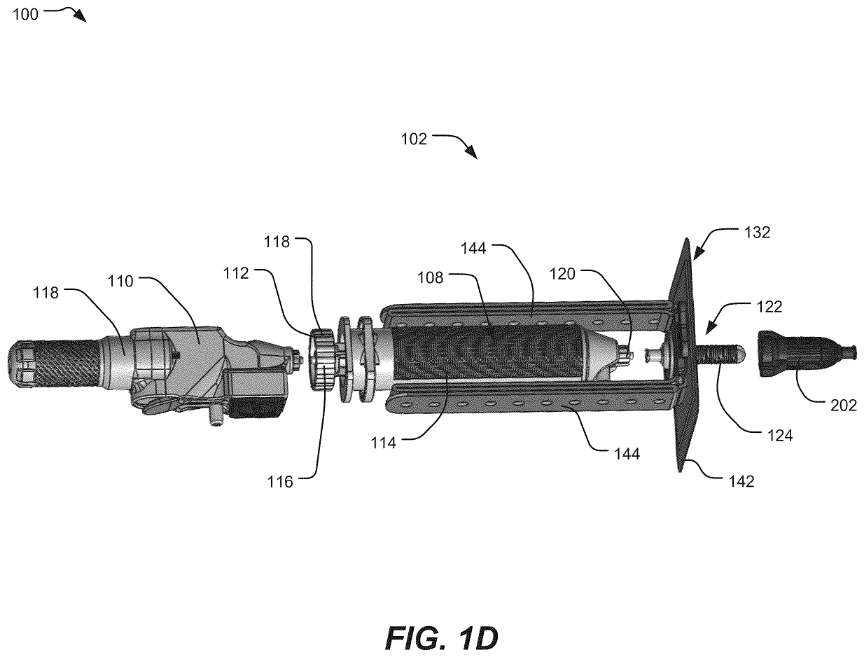

FIGS. 1A-1D illustrate an example system 100 including a portable wound treatment device 102. FIG. 1A shows the portable wound treatment device 102 in an assembled configuration 104 (e.g., for self-administration). FIG. 1B shows the portable wound treatment device 102 in an at least partly disassembled configuration 106. FIG. 1C shows the portable wound treatment device 102 with a compressed gas housing 108 and a carbon dioxide actuator 110. FIG. 1D shows the portable wound treatment device 102 with the compressed gas housing 108, the carbon dioxide actuator 110, and an air pump plunger 112. Each of these is discussed in greater detail below.

In some examples, the wound treatment device 102 includes the compressed gas housing 108 with an elongated body 114. The elongated body 114 can have a circular cross-sectional profile or other cross-sectional profiles, such as a triangular, square, or rectangular cross-sectional profiles. A first end 116 of the elongated body can include one or more components of a compressed gas actuator 118, which can include the carbon dioxide actuator 110, the air pump plunger 112, and/or combinations thereof. For instance, the first end 116 can include a connector (e.g., a threaded coupler) for connecting the first end 116 of the compressed gas housing 108 to the carbon dioxide actuator 110. Additionally or alternatively, first end 116 can include an opening for receiving the air pump plunger 112. In either configuration (e.g., or a combination configuration), the first end 116 can include the components to connect to an actuator which, upon being actuated by a user, causes compressed gas to flow through the compressed gas housing 108 from the first end 116 towards a second end 120.

In some instances, the wound treatment device 102 includes a balloon insertion assembly 122 which can couple to the second end 120 of the wound treatment device 102. The balloon insertion assembly 122 can include a tamponade balloon 124 which receives the compressed gas released from the compressed gas actuator 118 and, in response, at least partly inflates. The balloon insertion assembly 122 can also include a balloon cannula 126 with one or more positioning extensions 128. For instance, the positioning extension(s) 128 can extend radially outward from a balloon cannula housing 130, and can be used to secure the balloon insertion assembly 122 in place during a wound treatment procedure, as discussed in greater detail below.

In some examples, the wound treatment device 102 includes a device positioning assembly 132 which involves various components for securing the wound treatment device 102 in place at a site of a target wound being treated. The device positioning assembly 132 can mate with the balloon insertion assembly 122 and/or the compressed gas housing 108. For instance, the device positioning assembly 132 can include a locking balloon mount 134 with an opening 136 to receive the balloon cannula 126. A first positioning extension 137 can mate with a first side 138 of the locking balloon mount 134 (e.g., seated in an indent 140), and/or a second positioning extension 141 can mate with a second side of the locking balloon mount 134, such that the locking balloon mount 134 is sandwiched between the positioning extension(s) 128. The device positioning assembly 132 can further include an adhesive pad 142 (e.g., at the second side of the locking balloon mount 134) for adhering to a surface of the patient (e.g., skin and/or clothing) around the site of the target wound, to secure the device positioning assembly 132 in place at the target wound. Moreover, the wound treatment device 102 can include one or more strap(s) 144, extending from the locking balloon mount 134, and operable to wrap around a body part of the patient (e.g., a limb or portion of torso including the target wound). The technology disclosed herein can be used to deliver the tamponade balloon to a precise subcutaneous depth for immediate treatment which slows the bleeding until the patient can be transported to a clinic or emergency room.

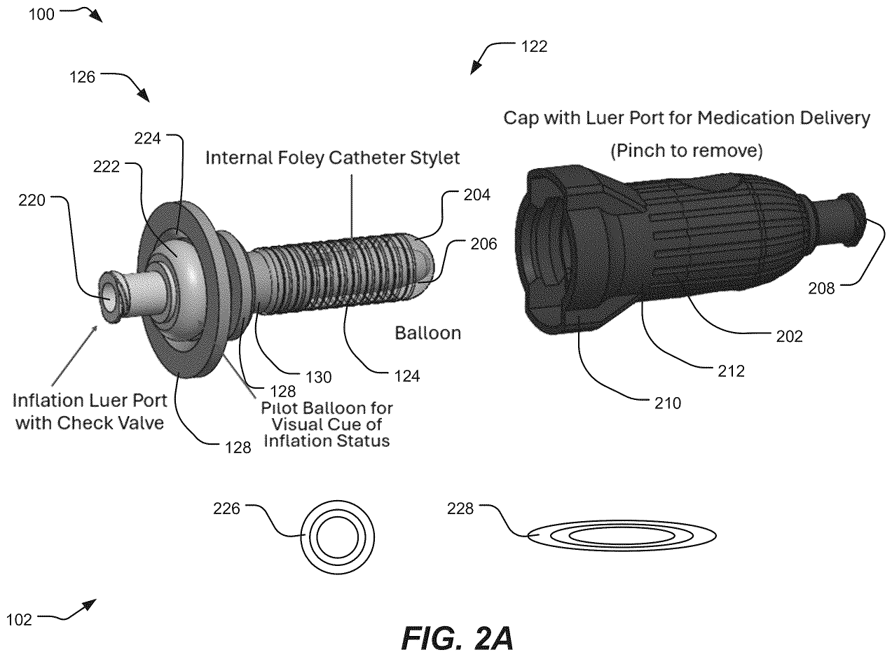

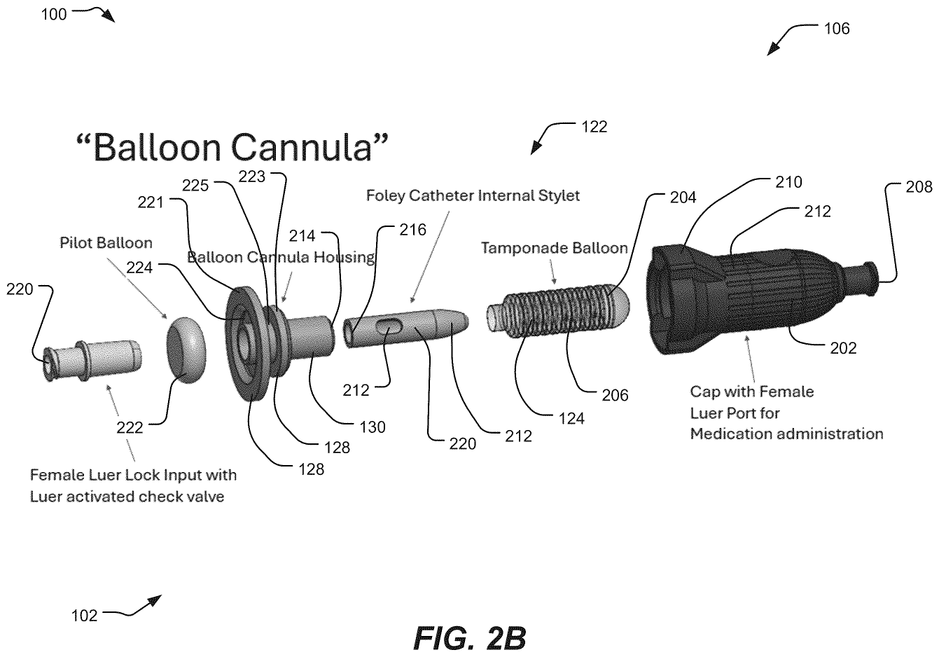

FIGS. 2A and 2B illustrate an example system 100 including the balloon insertion assembly 122 of the portable wound treatment device 102, which can form at least a portion of the system 100 depicted in FIGS. 1A-1D.

In some examples, the balloon insertion assembly 122 can include the tamponade balloon 124 and/or a cap 202 which can cover the tamponade balloon 124 prior to insertion into the target wound. The tamponade balloon 124 can include an inflatable and/or flexible balloon portion 204 with a wound treatment outer layer 206. The wound treatment outer layer 206 can be formed of various materials which fill the target wound and/or subdue bleeding from the target wound. For instance, the wound treatment outer layer 206 can be formed of an absorbent cloth, fabric, and/or mesh. Additionally or alternatively, the wound treatment outer layer 206 can include one or more carrier agents, such as a medication (e.g., an antibiotic, an anti-inflammatory agent, an anesthetic, a blood clotting agent, and so forth) and/or a sealant or adhesive (e.g., a mollusk-based glue). Furthermore, the cap 202 can include a luer port 208 for receiving and administering the carrier agent(s) to the tamponade balloon 124. Additionally, the cap 202 can include one or more gripping surfaces 210 which can extend from an outer surface 212 of the cap 202, such that pinching the gripping surface 210 causes the cap 202 to slightly deform and be removed from the tamponade balloon 124.

In some instances, the balloon insertion assembly 122 can include an internal foley catheter stylet 212 which extends from the balloon cannula housing 130. For instance, the balloon cannula housing 130 can include an end opening 214 configured to received an end 216 of the internal foley catheter stylet 212. An opening 218 formed into a side 220 (e.g., or an end) of the internal foley catheter stylet 212 can provide a pathway for the gas released from the compressed gas housing 108 into the tamponade balloon 124. In this way, an internal air pathway of the compressed gas housing 108 can be fluidly coupled to an internal air pathway of the balloon cannula housing 130 and the foley catheter internal stylet 212 such that the gas flows from one end of the wound treatment device 102 to the tamponade balloon 124, at least partially inflating the tamponade balloon 124.

Furthermore, the balloon cannula housing 130 can include the positioning extension(s) 128, such as a first positioning ring 221 extending radially outward from an outer surface of the balloon cannula housing 130, and/or a second positioning ring 223 extending radially outward from the outer surface of the balloon cannula housing 130. The first positioning ring 221 can be spaced apart from the second positioning ring 223 to form a gap 225 on the balloon cannula housing 130 and/or can be used to secure the balloon insertion assembly 122 in place during a wound treatment procedure, as discussed in greater detail below. The outer surface of the balloon cannula housing 130 at the gap 225 can be operable to mate with an inner edge of the opening 136 of the locking balloon mount 134, with the first positioning ring 221 on one side of the locking balloon mount 134, and the second positioning ring 223 on the other side of the locking balloon mount 134, thus securing the balloon cannula housing 130 in place within the locking balloon mount 134.

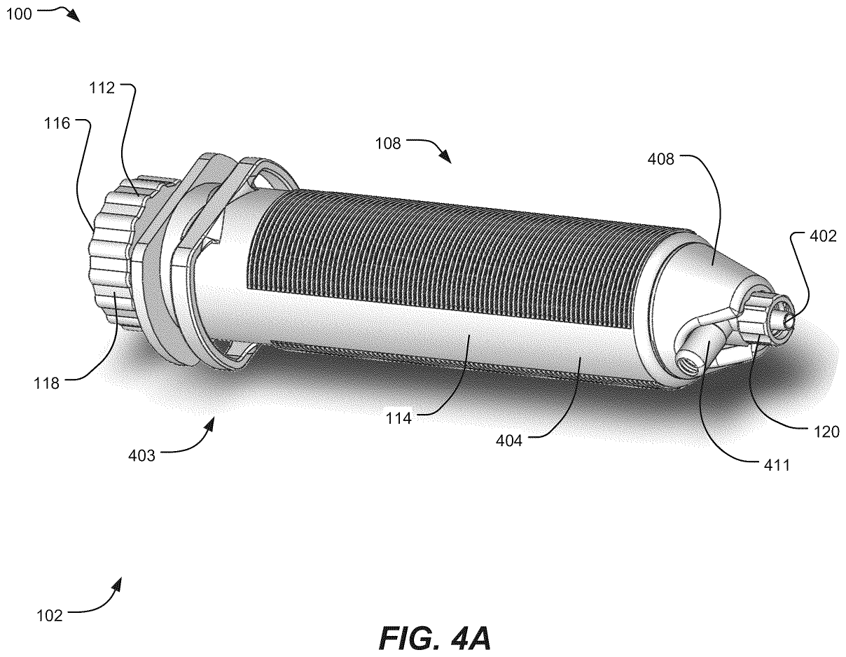

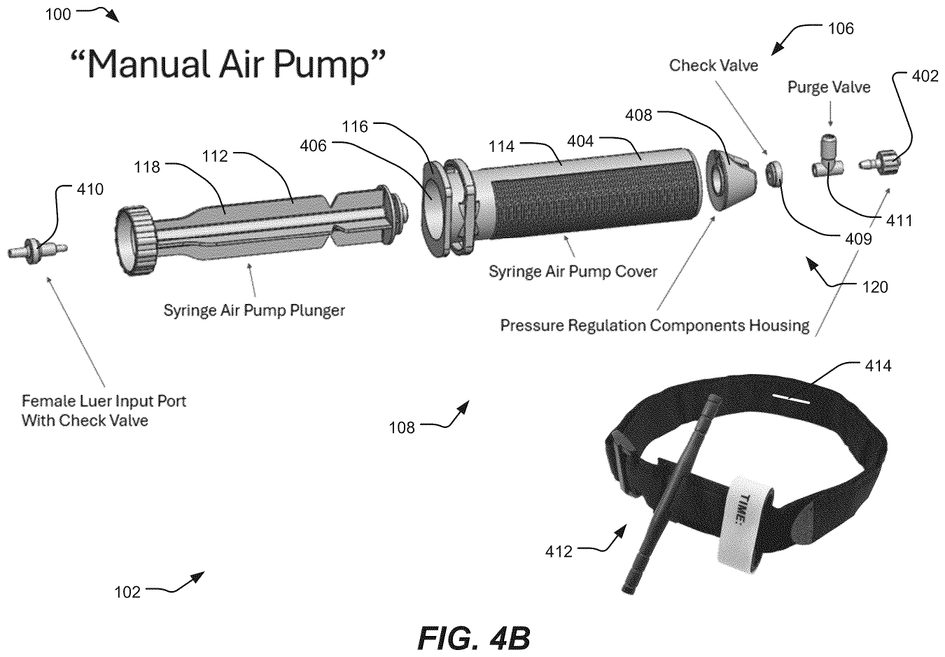

Additionally, the balloon insertion assembly 122 can include an inflation luer port 220 extending from an end of the balloon cannula housing 130. The inflation luer port 220 can include a female luer lock input and/or a luer-activated check valve. As such, the inflation luer port 220 can mate with a male luer output port 402 (e.g., depicted in FIGS. 4A and 4B) of the compressed gas housing 108 to create the air pathway into the tamponade balloon 124. Moreover, a pilot balloon 222 can be disposed in the balloon insertion assembly 122, for instance, in a recess 224 formed into an end of the balloon cannula housing 130. The pilot balloon 222 can at least partly wrap around the inflation luer port 220 and/or can be fluidly coupled to the air pathway of the wound treatment device 102. Upon actuating the compressed gas housing 108, gas can flow at least partly into the pilot balloon 222 to at least partially inflate the pilot balloon 222 and/or cause a change in shape of the pilot balloon 222. This change can operate as a visual indicator that the gas is flowing through the wound treatment device 102 and/or that the tamponade balloon 124 is inflating.

Moreover, any of the components of the balloon insertion assembly 122 disclosed herein can include a substantially circular cross-sectional profile 226, Additionally, in some examples, the balloon insertion assembly 122 can include another cross-sectional profile, such as a flattened and/or elongated cross-sectional profile 228. The balloon insertion assembly 122 can include an elongated and/or flattened balloon cannula housing 130, an elongated and/or flattened inflation luer port 220, an elongated and/or flattened pilot balloon 222, an elongated and/or flattened recess 224, an elongated and/or flattened foley catheter internal stylet 212, an elongated and/or flattened tamponade balloon 124, and/or any combination thereof to give the balloon insertion assembly 122 the flattened and/or elongated cross-sectional profile 228. In some scenarios, the balloon insertion assembly 122 with the flattened and/or elongated cross-sectional profile 228 can be used for certain types of wounds with a corresponding shape, such as knife wounds. The system(s) 110 disclosed herein can include a plurality of different balloon insertion assemblies 122 having the different cross-sectional profiles disclosed herein, and/or having different sizes, which can be selectively attached to the compressed gas housing 108 to match the type of wound being treated.

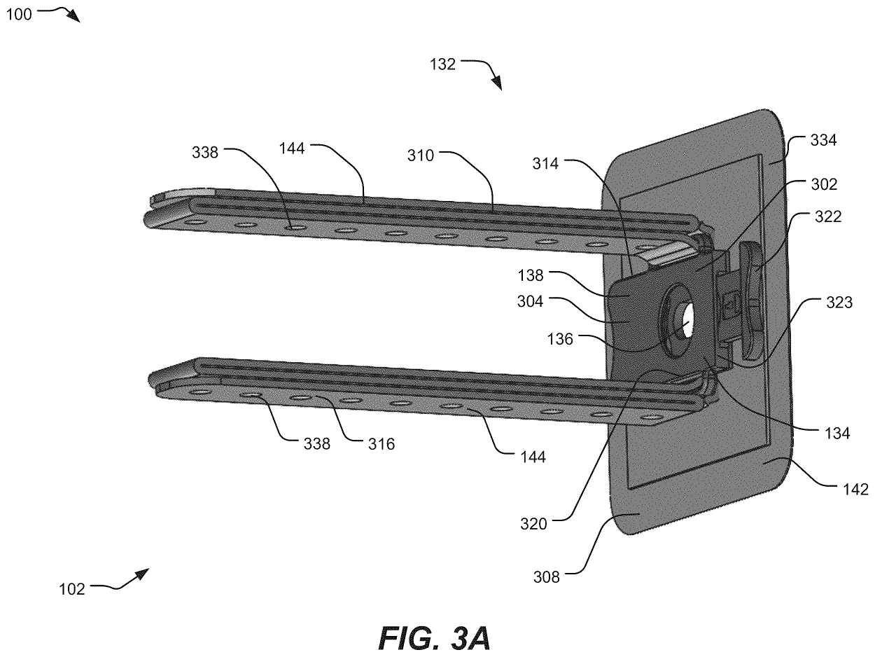

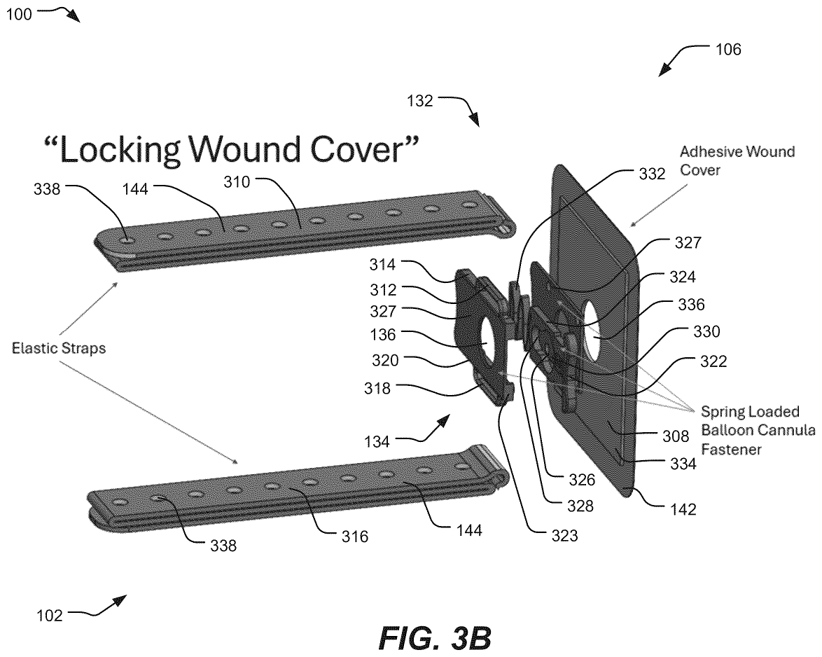

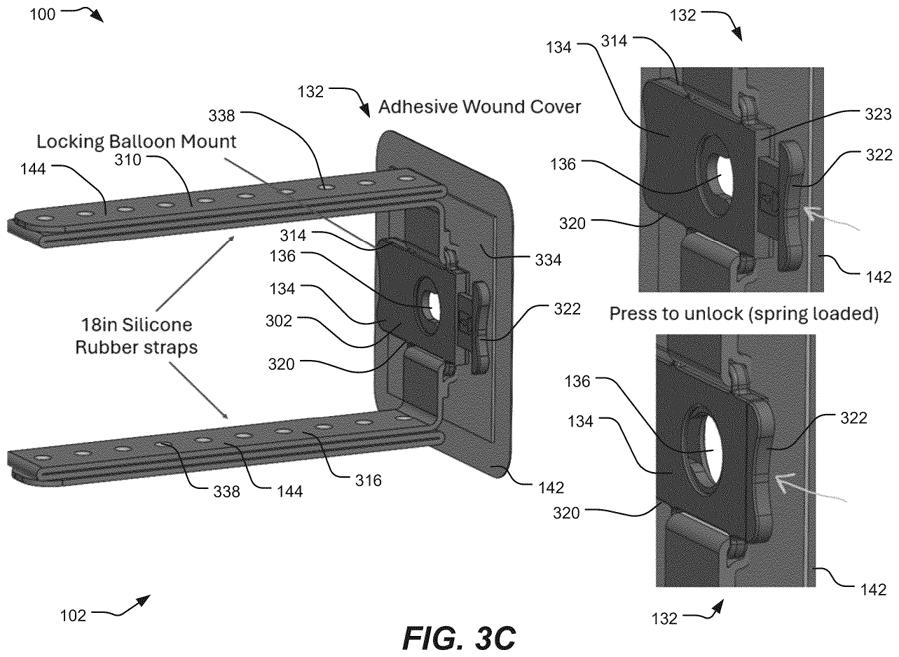

FIGS. 3A-3C illustrate an example system 100 including the device positioning assembly 132 of the portable wound treatment device 102, which can form at least a portion of the system(s) 100 depicted in FIGS. 1A-1D.

In some examples, the device positioning assembly 132 includes the locking balloon mount 134, the adhesive pad 142, and/or the strap(s) 144. For instance, the locking balloon mount 134 can include a substantially rigid body 302 with the opening 136 for receiving the balloon insertion assembly 122 formed into a middle portion of the rigid body 302. The locking balloon mount 134 can be substantially flat with a first face 304 directed at the compressed gas housing 108, and/or a second face abutting a non-adhesive side 308 of the adhesive pad 142. A first strap 310 can couple to a first loop 312 at a first side 314 of the locking balloon mount 134, and/or a second strap 316 can couple to a second loop 318 at a second side 320 of the locking balloon mount 134.

Furthermore, the locking balloon mount 134 can include a spring-loaded release tab 322. The spring-loaded release tab can extend from a third side 323 of the locking balloon mount 134, which can be between the first side 314 and the second side 320. The spring-loaded release tab 322 can form a part of an inner sliding portion 324 of the locking balloon mount 322. The inner sliding portion 324 can include an opening 326 that aligns with openings of outer portions 327 of the locking balloon mount 134 to form the opening 136 for receiving the balloon insertion assembly 122. The opening 326 of the inner sliding potion 324 can include a first opening diameter 328 merged with a second opening diameter 330. The first opening diameter 328 can be smaller than the second opening diameter 330, such that moving/sliding the inner sliding portion 324 causes an expansion and/or restriction of the opening 136 (e.g., by shifting between the first opening diameter 328 and the second opening diameter 330). The inner sliding portion 324 can also include a spring 332 (e.g., a living spring) which can extend from a side opposite the spring-loaded release tab 322. The spring 332 can abut an inner wall of the locking balloon mount 134 to create a tensional force in the spring-loaded release tab 322. Additionally, the flat body 302 of the locking balloon mount 134 can have one or more or all dimensions(s), such as a first width dimension and a first length dimension, being less than one or more or all dimension(s) of the adhesive pad 142, such as a second width dimension and/or a second length dimension. In this way, the adhesive pad 142 can have a surface area 334 that is substantially larger than the locking balloon mount 134. The adhesive pad 142 can also include an opening 336 which aligns with the opening 136 of the locking balloon mount 134, such that the balloon insertion assembly 122 can pass through the adhesive pad 142.

In some examples, the device positioning assembly 132 can include one or more strap(s) 144, such as the first strap 310 and/or the second strap 316. These strap(s) 144 can be formed of a flexible and/or elastic material and can be transitioned from a stowed position to a functioning position during use of the wound treatment device. Along with the locking balloon mount 134 and/or the adhesive pad 142, the strap(s) 144 can operate to secure the wound treatment device 102 in place at the site of the target wound. For instance, in scenarios where the site of target wound includes a limb (e.g., an arm or a leg), the strap(s) 144 can wrap around the limb and be adjoined together, for instance, using one or more holes 338 and/or a strap locking mechanism. In scenarios where the site of the target wound includes a torso, the strap(s) 144 can wrap around the torso. Also, additionally or alternatively to securing the wound treatment device 102, the strap(s) 144 can be used to provide pressure to the target wound, like a tourniquet, which can slow the bleeding at the target wound.

FIGS. 4A and 4B illustrate an example system 100 including a wound treatment device 102 with the compressed gas housing 108. As depicted in FIGS. 4A and 4B, the compressed gas housing 108 can include an air pump assembly 403, which can form at least a portion of the system 100 depicted in FIGS. 1A-1D.

In some examples, the compressed gas housing 108 includes an outer housing 404 which can include a cover for the air pump plunger 112. The air pump plunger 112 can be extendible from the first end 116 of the compressed gas housing 108 and, to actuate the wound treatment device 102, the air pump plunger 112 can be slid and/or pushed into an opening 406 at the first end 116. The second end 120 of the compressed gas housing 108 can include a pressure regulation components housing 408. The pressure regulation components housing 408 can include an outer surface (e.g., with a tapered width) which at least partially surrounds a check valve 409, a purge valve 411, and/or the male luer output port 402. Additionally, the compressed gas housing can include a female luer input port 410 (e.g., with a check valve) disposed at the first end 116. The female luer input port 410 can be operable to mate with the carbon dioxide actuator 110. As such, the compressed gas housing 108 can have multiple options for providing the gas to the tamponade balloon 124 during an inflation procedure, including manually sliding the air pump plunger 112 towards the second end 120, and/or actuation of the carbon dioxide actuator 110 to release pressurized carbon dioxide from a carbon dioxide canister.

Furthermore, in some instances, the system 100 can include a Combat Applications Tourniquet (CAT) 412, which can be accessed and/or unfurled by the patient self-administering the wound treatment device 102, and/or by another person, such as a field medic, assisting with the wound treatment. Also, a slit 414 can be cut into the CAT 414 (e.g., in the field during treatment and/or prior to treatment) such that the CAT can be placed over the wound treatment device 102 with the actuation components of the wound treatment device 102 accessible through the slit 414.

Additionally, the occlusion balloon can be part of the balloon cannula. The balloon can be designed to inflate from a distal to proximal orientation in the wound. This is done for the following: a) First, distal to proximal can allow the balloon to “anchor the device” in the wound so that it doesn't “auto eject” from the wound as it inflates. A proximal to distal inflation orientation causes proximal pressures to work the wound treatment device 102 out of the wound as it inflates. b). Second, by inflating distal to proximal, this can force immediate pressure forward in the wound channel and causes a direct tamponade on the damaged vessels and tissues of the wound. This is described as creating an “internal tourniquet” on the wound and can help eliminate the need to use an external tourniquet. Additionally, by creating an internal tourniquet on the wound, the device allows for peripheral vascular flow to be maintained and doesn't promote atrophy of the limb due to loss of oxygen.

Furthermore, the balloon can have manual or automated inflation. The occlusion balloon can be inflated through a manual or automated methodology. Also, the wound treatment device 102 disclosed herein can be single use. The wound treatment device 102 can be designed to be a single use device and can be discarded after the initial use. Once the balloon has been inflated and is set under pressure, it can be designed to remain in the wound until a surgeon or medical technician can deflate and dispose of the device. Additionally, the wound treatment device 102 can be a sterile device. The balloon cannula and especially the surgical occlusion balloon can be packaged and shipped sterile. Once the balloon and the cannula have been removed from their protective, sterile packaging, the device can be considered “utilized” regardless if the device is actually deployed or not.

Additionally, the systems 100 disclosed herein can include a device recovery program. Since the wound treatment device 102 can be delivered to the end user in a sterile, protective manner, it means it can carry a two year sterile shelf life with an expiration date stamped on the packaging and the device itself. Once the wound treatment device 102 has reached its two year sterile shelf life limitation, it may be considered expired and no longer supported by the company's protective sterility warranties. Therefore, unused product recovery program may be deployed. This program can allow for an entity to recover unused product that has expired and replace it with brand new product with a full 2 year sterility warranty. The entity can take these unused devices and repurpose the recoverable parts into new products. The unrecoverable parts may be discarded.

Also, the technology disclosed herein can include a device insertion methodology, such as the process of the steps taken to deploy the wound treatment device 102 into a wound and a body. Additionally, the technology can include a balloon expansion methodology, including the dynamics surrounding balloon inflation. The system 100 can also include particular internal wound inflation pressure ranges. For instance, the inflation pressure range of the balloon can be anywhere from 0.5 lbs per square inch to 12 lbs per square inch, internal occlusion pressure inside the wound. Another range can be being 2 lbs per square inch to 5 lbs per square inch internal balloon pressure. Moreover, the technology can include balloon compliance in that the occlusion balloon can take on a “compliant Shape” or a “Non-Compliant” shape. A non-compliant balloon may allow the balloon to expand to fit the internal shape of the wound. Also, as noted above, the balloon can have a particular balloon inflation direction. the balloon can be designed to inflate distal end to proximal end according to a balloon inflation methodology.

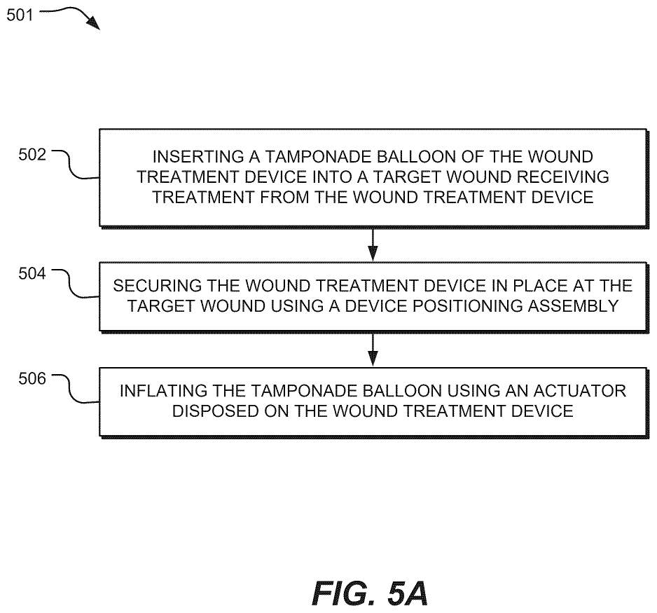

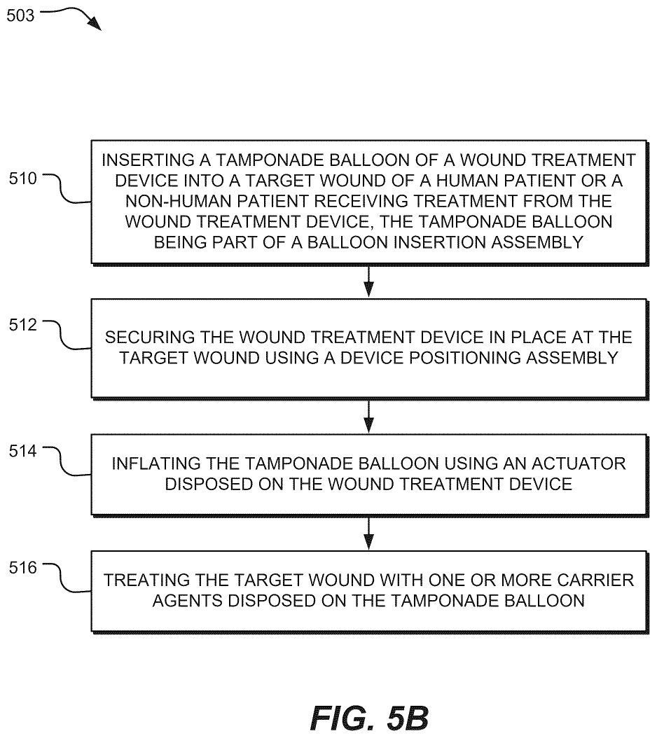

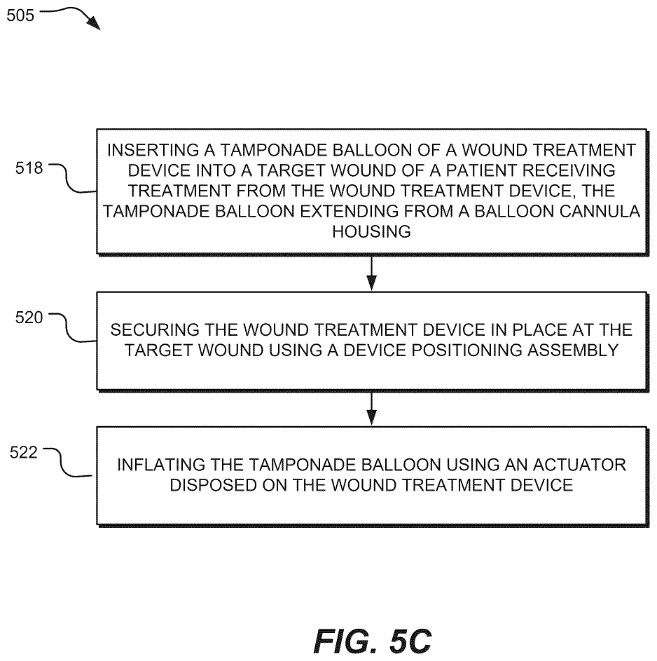

FIGS. 5A-5C illustrate example method 501, method 503, and method 505 to treat a wound which can be performed by any of the system(s) 100 depicted in FIGS. 1A-4B.

For example, as shown in FIG. 5A at operation 502, the method 501 can insert a tamponade balloon of the wound treatment device into a target wound receiving treatment from the wound treatment device. At operation 504, the method 501 can secure the wound treatment device in place at the target wound using a device positioning assembly. At operation 506, the method 501 can inflate the tamponade balloon using an actuator disposed on the wound treatment device.

Turning to FIG. 5B, at operation 510, the method 503 can insert a tamponade balloon of a wound treatment device into a target wound of a human patient or a non-human patient receiving treatment from the wound treatment device, the tamponade balloon being part of a balloon insertion assembly. At operation 512, the method 503 can secure the wound treatment device in place at the target wound using a device positioning assembly. At operation 514, the method 503 can inflate the tamponade balloon using an actuator disposed on the wound treatment device. At operation 516, the method 503 can treat the target wound with one or more carrier agents disposed on the tamponade balloon.

Furthermore, as shown in FIG. 5C, at operation 518, the method 505 can insert a tamponade balloon of a wound treatment device into a target wound of a patient receiving treatment from the wound treatment device, the tamponade balloon extending from a balloon cannula housing. At operation 520, the method 505 can secure the wound treatment device in place at the target wound using a device positioning assembly. At operation 522, the method 505 can inflate the tamponade balloon using an actuator disposed on the wound treatment device.

It is to be understood that the specific order or hierarchy of steps in the method(s) 501, 503, and 505 depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A method of treating a wound comprising:
inserting a tamponade balloon of a wound treatment device into a target wound receiving treatment from the wound treatment device;
securing the wound treatment device in place at the target wound using a device positioning assembly; and
inflating the tamponade balloon using an actuator disposed on the wound treatment device.

2. The method of claim 1,
wherein,
the target wound occurs in a non-human animal.

3. The method of claim 2,
wherein,
the non-human animal includes a cow or bull.

4. The method of claim 2,
wherein,
the target wound is a result of a fly worm infection.

5. The method of claim 4,
wherein,
the tamponade balloon is coated in one or more carrier agents to treat the fly worm infection.

6. The method of claim 5,
wherein,
the one or more carrier agents include at least one of a pesticide, a larvicide, a macrocyclic lactone, or an anthelmintic.

7. The method of claim 2,
wherein,
the non-human animal includes one of cattle, a sheep, a goat, a pig, a dog, a cat, a chicken, a horse, a donkey, a duck, a buffalo, a llama, an alpaca, or a camel.

8. The method of claim 2,
wherein,
the inflating of the tamponade balloon causes one or more treatment agents to be provided into the target wound.

9. The method of claim 8,
wherein,
the treatment agent at least one of reduces or prevents a fly worm infection.

10. The method of claim 1,
wherein,
the wound treatment device includes a balloon cannula housing with an elongated cross-sectional profile corresponding to a shape of the target wound.

11. The method of claim 1,
wherein,
the wound treatment device includes a female luer locking port, and
the method further includes administering one or more insecticide or larvicide to the tamponade balloon through the female luer locking port.

12. A method of treating a wound comprising:
inserting a tamponade balloon of a wound treatment device into a target wound of a human patient or a non-human patient receiving treatment from the wound treatment device, the tamponade balloon being part of a balloon insertion assembly;
securing the wound treatment device in place at the target wound using a device positioning assembly;
inflating the tamponade balloon using an actuator disposed on the wound treatment device; and
treating the target wound with one or more carrier agents disposed on the tamponade balloon.

13. The method of claim 12,
wherein,
the target wound is of the non-human patient, and
the one or more carrier agents includes an insecticide.

14. The method of claim 12,
wherein,
the target wound is of the non-human patient, and
the one or more carrier agents includes a larvicide.

15. The method of claim 12,
wherein,
the target wound is a knife wound of the human patient.

16. The method of claim 15,
wherein,
the balloon insertion assembly includes an elongated cross-sectional profile which corresponds to a cross-sectional profile of the knife wound.

17. A method of treating a wound comprising:
inserting a tamponade balloon of a wound treatment device into a target wound of a patient receiving treatment from the wound treatment device, the tamponade balloon extending from a balloon cannula housing;
securing the wound treatment device in place at the target wound using a device positioning assembly; and
inflating the tamponade balloon using an actuator disposed on the wound treatment device.

18. The method of claim 17,
wherein,
the balloon cannula housing includes an elongated cross-sectional profile.

19. The method of claim 18, further comprising:
selecting the balloon cannula housing including the elongated cross-sectional profile from a plurality of balloon cannula housings having different cross-sectional profiles, the selecting of the balloon cannula being based on a shape of the target wound.

20. The method of claim 17,
wherein,
the target wound includes an insect or parasite infection, and
the inflating of the tamponade balloon treats the insect or parasite infection.
